(19) United States
(12) Patent Application Publication
(10) Pub. No.: US 2006/0005931 A1
(43) Pub. Date: Jan. 12, 2006

(54) Label removing tool

(76) Inventor: Rocky Sherman, Huntington Beach, CA (US)

Correspondence Address: Law Offices of Dennis W. Beech, P.O. Box 519, Murrietta, CA, 92564, US

(21) Appl. No.: 11/228,701

(22) Filed: Sep. 15, 2005

Related U.S. Application Data
(63) Continuation-in-part of application No. 10/840,500, filed on May 6, 2004.

(51) Int. Cl. B25B 7/00; A61B 17/50; B25B 9/00

(52) U.S. Cl. 156/584; 156/344; 294/99.2; 606/210

(57) ABSTRACT

The label removing tool may be used to remove adhesive labels from objects. A bit may have two prongs positioned adjacent and parallel to each other and attached at a proximal end. One of said prongs may be shaped at a distal end to aid in insertion under a label. A holding tool may have a cavity therein for insertion of the proximal end with a fastener to retain the bit in the holding tool.

This application is a continuation-in-part of U.S. patent application Ser. No. 10/840,500 filed on May 6, 2004, which application is pending.

### BACKGROUND OF THE INVENTION

This invention relates to devices for removal of adhesive labels or decals from objects to which they may be attached. The new tool may have a pair of rods with shaped distal ends to engage a portion of a label attached to an object. The tool may then be rotated by a rotational device to peel the label from the object.

Labels and decals currently may be removed from an object such as a football helmet by stripping, scraping, peeling and the like the adhesive label from the surface of the helmet. Methods that generally may have been used may include use of a sharp scraping blade or tool to repetitively scrape against the ruminants of a label edge to scrape portions of a label off the helmet. There may also be machines for removal of labels from bottles or other containers that use high pressure fluid impact against attached labels for removal thereof.

### SUMMARY OF THE INVENTION

The present invention is directed to devices used to remove adhesive labels from objects. A bit may have two prongs positioned adjacent and parallel to each other and attached at a proximal end. One of said prongs may be shaped at a distal end to aid in insertion under a label. A holding tool may have a cavity therein for insertion of the proximal end with a fastener to retain the bit in the holding tool.

These and other features, aspects and advantages of the present invention will become better understood with reference to the following drawings, description and claims.

### BRIEF DESCRIPTION OF THE DRAWINGS

FIG. 1 illustrates a perspective view of a football helmet and application of the label removal tool to labels according to an embodiment of the invention;

FIG. 2 illustrates a perspective view of the removal tool rolling a label according to an embodiment of the invention;

FIG. 3 illustrates a side exploded view of the removal tool according to an embodiment of the invention;

FIG. 4 illustrates a side exploded view of the removal tool according to an embodiment of the invention.

### DETAILED DESCRIPTION

The following detailed description represents the best currently contemplated modes for carrying out the invention. The description is not to be taken in a limiting sense, but is made merely for the purpose of illustrating the general principles of the invention.

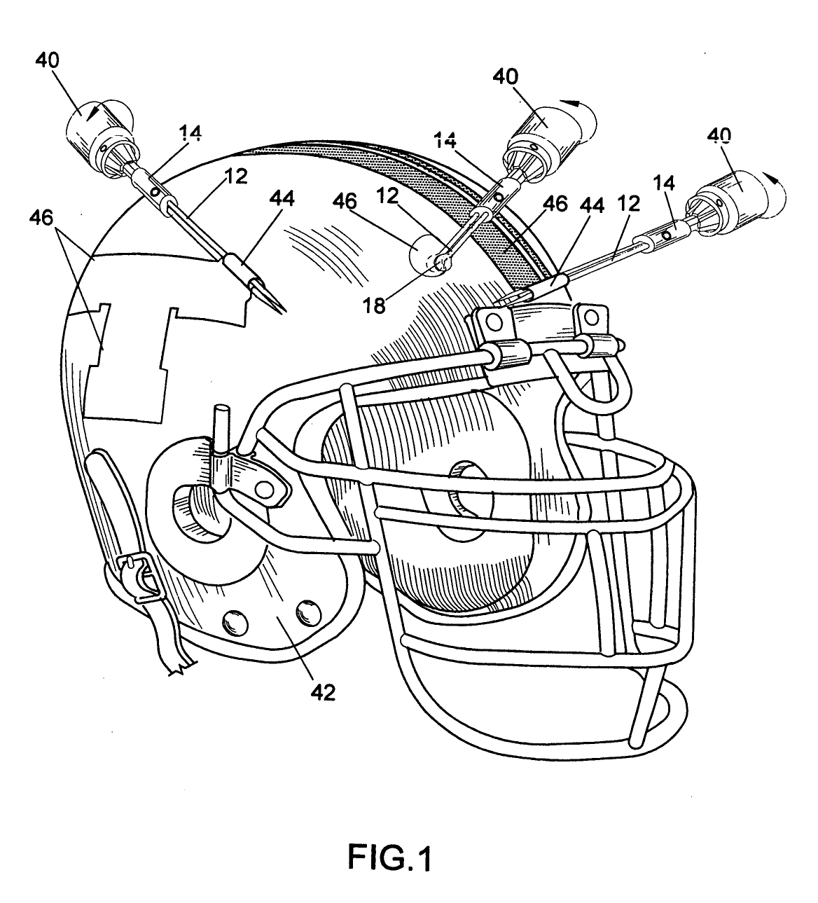

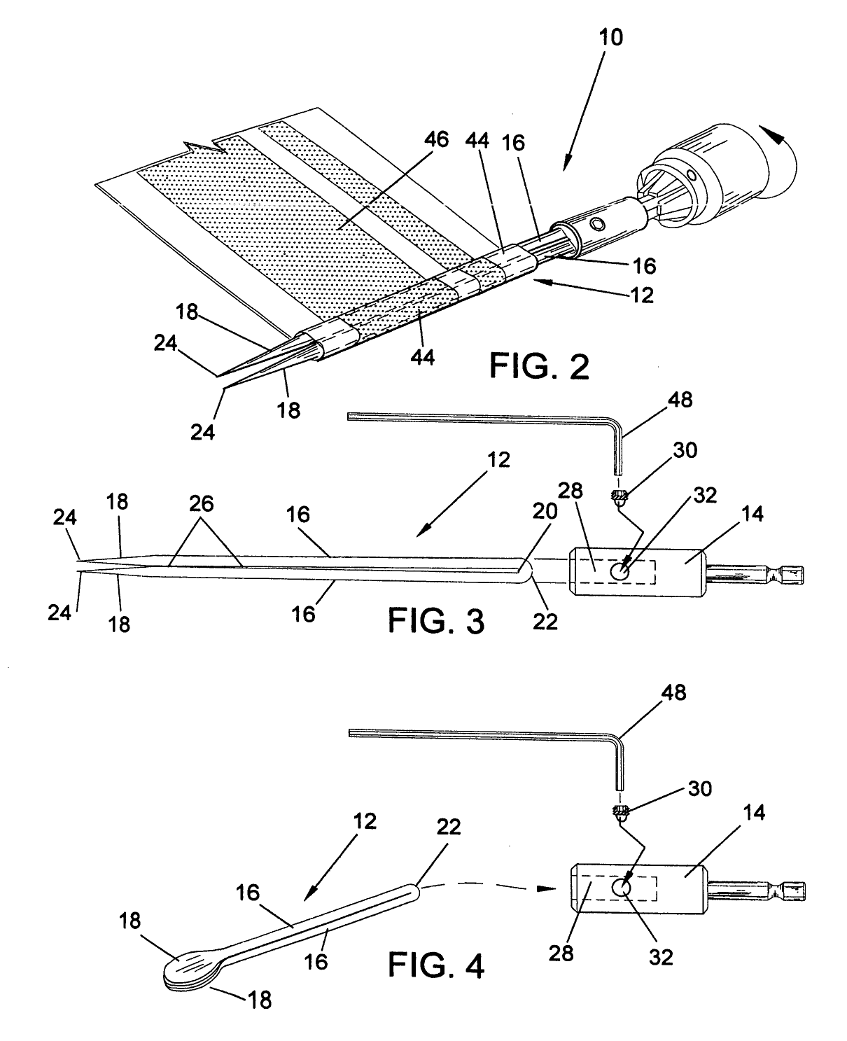

Referring to FIGS. 1 and 2, the label removal tool 10 may have a bit 12 that may generally have a fork shape and may be attached to a holding tool 14. The holding tool 14 may be insertable in and retainable by a chuck 40 of a rotational device (not shown) such as a hand drill, power hand drill or motorized rotational tool. The bit 12 may have two prongs 16 positioned adjacent and parallel to each other. The prongs may have tapered or pointed distal ends 18 or may be flattened to a disk, rectangular or other shape for insertion under a label 46 or football decal sticker attached to a helmet 42 or other structure. The power or momentum of a rotational tool may rotate the label 46 against the adhesive holding force to roll the label 46 onto the bit 12 and off of an object such as a football helmet.

In use one prong 16 may be pushed, slid or inserted under a portion 44 or edge of a label 46, decal, sticker or other adhesive attached element. A rotational device may then be operated to rotate the chuck 40 to rotate the prongs 16 to roll or remove a label 46 from a helmet 42.

Referring to FIGS. 2 and 3, the bit 12 may be formed from a single rod of material such as stainless steel or other material that may be rigid yet springy enough to allow sliding of a portion of a label 46 between the two prongs 16 when they are biased one against the other. A bent rod may form two prongs 16 with a bend portion 20 therebetween at a proximal end 22. The two prongs 16 may be tapered at their distal end 18 to form a pointed tip 24 that may be spaced apart to aid in insertion under an adhesive label 46. Prongs 16 may be formed from different length rods.

The bit 12 may be inserted into a cavity 28 in the holding tool 14 and retained therein by a screw 30 threadably inserted in aperture 32 by a threading tool 48 such as an Allen wrench. A screw 30 having a tapered end may be used to engage the bit 12 at the proximal end 22. The holding tool may then be inserted in the chuck 40 and retained as generally understood for chucks of rotating apparatus. The two prongs 16 may be bias against each other along a longitudinal portion 26 of their shaft adjacent tips 24. When a portion of a label may be pushed between the prongs 16, the label portion may be gripped by the force of the two touching longitudinal portions 26 of the prongs 16. This may aid in rolling up and peeling off of an adhesive label form an object.

Referring to FIG. 4, a bit 12 may have flattened distal ends 18 that may be disk shape, rectangular or other shape for insertion under and gripping of a label or sticker. The distal ends 18 may be biased against each other to provide a gripping force against a label pushed between the distal ends 18. This may aid in removal of relatively small labels or stickers. The bit 12 may be attached to the holding tool 14 as described above. While the bits 12 have been described as being formed from a single bent rod, other methods of manufacture and attachment of two or more prongs 16 may be used to form the bit 12, for example, welding or other methods to attach prongs 16 one to another.

While the invention has been particularly shown and described with respect to the illustrated embodiments thereof, it will be understood by those skilled in the art that the foregoing and other changes in form and details may be made therein without departing from the spirit and scope of the invention.

I claim:

1. A device for removal of adhesive labels from objects comprising:
a bit having two prongs positioned adjacent and parallel to each other and attached at a proximal end wherein one of said prongs is shaped at a distal end to space apart a tip of each of said prongs and to aid in insertion under a label;
said prongs are biased one against another along a longitudinal portion of each adjacent said tip of each of said prongs; and
a holding tool having a cavity therein for insertion of said proximal end with a fastener to retain said bit in said holding tool.

2. The device as in claim 1 wherein said bit is formed from a rod bent at an approximate middle to form said proximal end.

3. The device as in claim 1 wherein a distal end of each prong is tapered to form a pointed tip.

4. The device as in claim 1 wherein a distal end of each prong is flattened and a flattened portion of each prong is biased one against another.

5. The device as in claim 1 wherein said fastener is a screw threadably disposed in an aperture of said holding tool to engage said proximal end.

6. The device as in claim 1 wherein said holding tool attachable to a rotational tool having sufficient power to rotate said device against an adhesive holding force of said adhesive labels.

7. The device as in claim 1 wherein said prongs are generally a straight cylindrical shape and said tips have a tapered point at an approximate radial center of said prongs.

8. A method for removal of adhesive labels from objects comprising:
attaching a label removing tool to a rotational device;
inserting one prong of a plurality of adjacent prongs of said label removing tool between an object and an adhesive label attached thereto to grip said adhesive label; and
activating said rotational device to rotate said adhesive label about said plurality of adjacent prongs.

9. The method as in claim 8 wherein said label removing tool having two adjacent prongs.
